(19) United States
(12) Patent Application Publication
(10) Pub. No.: US 2011/0252665 A1
(43) Pub. Date: Oct. 20, 2011

(54) SOFT AND ELASTIC SHOE PAD

(75) Inventor: Ching-Tao TSAI, Taipei (TW)

(73) Assignee: FUSCO INDUSTRIAL CORPORATION, Taipei (TW)

(21) Appl. No.: 12/759,859

(22) Filed: Apr. 14, 2010

(51) Int. Cl. A43B 13/38; A61F 5/14

(52) U.S. Cl. 36/44; 36/147

(57) ABSTRACT

A shoe pad includes a soft and elastic layer and an air permeable layer connected onto the soft and elastic layer. The soft and elastic layer includes a front metatarsal section, a mid foot section and a rear heel section. Central portions of the front metatarsal section and the rear heel section have a plurality of cavities. The cavities are discrete from each other for enhancing the structural strength of the shoe pad. The mid foot section has a thickened arch portion, and the arch portion has a plurality of reinforcement structures for enhancing the supporting strength. The periphery portion of the rear heel section is thicker than the central portion of the rear heel section, so when a foot heel steps on the rear heel section, a feeling of stable descending is provided.

### BACKGROUND OF THE INVENTION

1. Field of the Invention

The present invention relates to a shoe pad capable of being placed inside a shoe, an in particular to a soft and elastic shoe pad having functions of shock absorbing and keeping the normal walking gait.

2. The Prior Arts

When a man is walking, each foot is subject to a force which is 0.8-1.2 times of the body weight, not to mention the reaction force, which directly impacts the foot sole, knee and spine. Thus, a pad is often provided for absorbing the impact. Otherwise, many foot-related problems may occur in a long term period.

A soft plastic shoe pad is an insert type shoe pad. The shoe pad is not a fixed type shoe pad, therefore can be taken out of the shoe to be placed in another one. The soft plastic shoe pads are viscous and retractable. Due to the property of viscosity, the soft plastic shoe pads can absorb impacts to protect the feet. The reason why the soft plastic shoe pads are popular is that they can be fabricated thin enough to be placed inside the shoes. For providing comfortable and soft feelings, a fabric capable of absorbing moisture and breathing is often connected on the top surface of the soft plastic shoe pad.

However, the shock absorbing property of the soft plastic shoe pad has a disadvantage. Because the soft plastic shoe pad has a damping effect, the user requires more strenuous effort to walk and muscles are therefore easier to fatigue. Therefore, how to take advantage of the shock absorbing property of soft plastic shoe pad and improve the structure of shoe pad to ensure healthy walking gait without taking too much laborious effort when wearing the shoe pad is needed to be solved.

### SUMMARY OF THE INVENTION

A primary objective of the present invention is to provide a soft and elastic shoe pad capable of absorbing impacts and providing an elastic forward moving assistance during walking.

Another objective of the present invention is to provide a soft and elastic shoe pad which is ergonomic for solving the problem that muscle fatigue is easily generated when conventional soft plastic shoe pads are worn.

In order to achieve the objectives mentioned above, a soft and elastic shoe pad according to the present invention includes a soft and elastic layer and an air permeable layer connected on a surface of the soft and elastic layer. The soft and elastic layer includes a front metatarsal section, a mid foot section and a rear heel section. Central portions of the front metatarsal section and the rear heel section at the bottom surface of the soft and elastic layer includes a plurality of cavities for enhancing the structural strength at parts of the shoe pad that are often in a bended status. The mid foot section has a thickened arch portion, and the arch portion includes a plurality of reinforcement structures for enhancing the supporting strength. The periphery portion of the rear heel section is thicker than the central portion of the rear heel section. Thus, when the foot heel is stepped on the rear heel section, a feeling of descending is generated, and the inner and outer rotation angles of the foot heel during walking can be effectively and stably controlled.

Beside the mentioned functions provided by the present invention, the soft and elastic shoe pad according to the present invention is made of a soft and elastic material having viscosity property, e.g. silicone gel, latex, rubber, PU, TPE, EVA, TPR, or non-toxic thermoplastic, etc. So the shoe pad not only can effectively absorb the ground reacting force, but also protect the foot from impact and injury. The thickened arch portion ensures a proper support when the foot steps on the ground, and a function of assisting elastic forward moving is provided. Therefore, the walking movement can be achieved without over using muscle strength, and the muscle fatigue is greatly reduced.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a perspective bottom view showing a soft and elastic shoe pad according to a first preferred embodiment of the present invention;

FIG. 2 is an exploded view showing the soft and elastic shoe pad according to the first preferred embodiment of the present invention;

FIG. 3 is a bottom view showing the soft and elastic shoe pad according to the first preferred embodiment of the present invention;

FIG. 3A is a cross sectional view taken along line 3A-3A of FIG. 3;

FIG. 3B is a cross sectional view taken along line 3B-3B of FIG. 3;

FIG. 3C is a cross sectional view taken along line 3C-3C of FIG. 3;

FIG. 4 is an exploded view showing the soft and elastic shoe pad according to a second preferred embodiment of the present invention;

FIG. 5 is a bottom view showing the soft and elastic shoe pad according to the second preferred embodiment of the present invention;

FIG. 5A is a cross sectional view taken along line 5A-5A of FIG. 5;

FIG. 5B is a cross sectional view taken along line 5B-5B of FIG. 5; and

FIG. 6 is a side view showing a foot stepping on the shoe pad according to the present invention.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

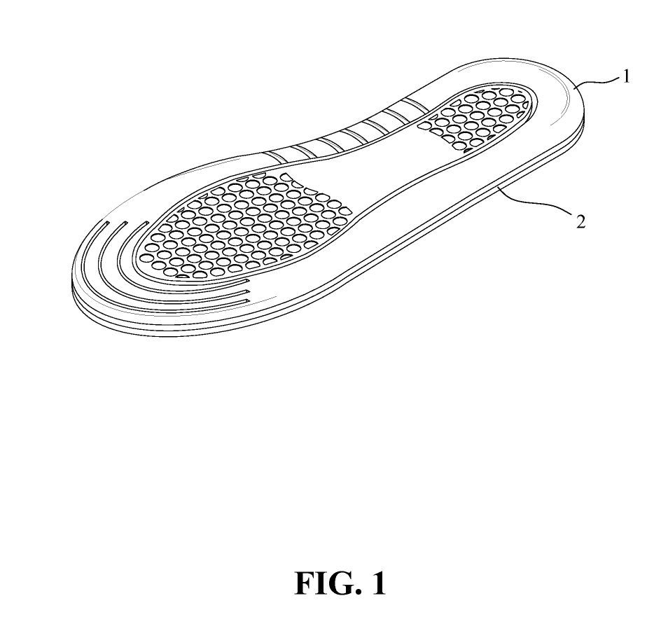

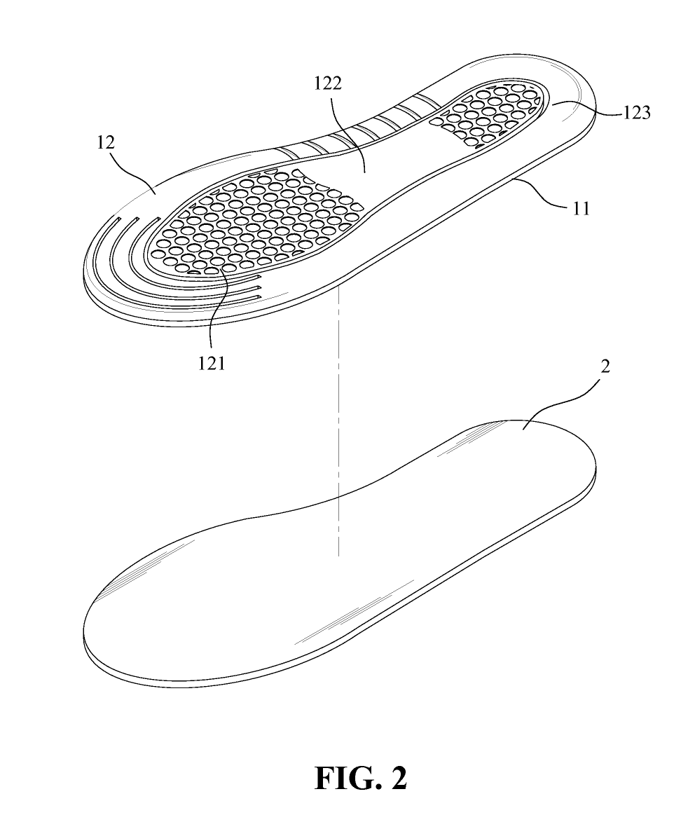

A shoe pad according to the present invention is disposed in a shoe. Referring to FIGS. 1 and 2, the soft and elastic shoe pad according to a first embodiment of the present invention includes a soft and elastic layer 1 and an air permeable layer 2. Because the design for the left foot is symmetrical to that for the right foot, hereinafter a shoe pad for the right foot is adopted for illustration.

The soft and elastic layer 1 may be made of a soft and elastic material, including but not limited to silicone gel, latex, rubber, PU, TPE, EVA, or combination thereof. The soft and elastic layer 1 is integrally formed and is provided with effects of pressure relieving and shock absorbing. The soft and elastic layer 1 has a shape corresponding to the outline of human's foot, and includes a top surface 11 and a bottom surface 12. The top surface 11 is associated with the air permeable layer 2. The shape of the air permeable layer 2 is tailored corresponding to the shape of the soft and elastic layer 1. The air permeable layer 2 is made of fabrics that are soft, comfortable, air permeable, and capable of absorbing moisture. The soft and elastic layer 1 includes, with respect to the structure of the foot, a front metatarsal section 121, a mid foot section 122 and a rear heel section 123. The front metatarsal section 121 is served to be stepped by the forefoot; the mid foot section 122 is served to be in contact with the mid portion of the foot including the arch of the foot; the rear heel section 123 is served to be stepped by the foot heel. A front edge of the front metatarsal section 121 of the soft and elastic layer 1 are designed according to the curve defined by toes. The shoe pad is gradually thickened from the rear end of the front metatarsal section 121 towards a rear edge of the rear heel section 123, and therefore the shoe pad is capable of being placed and retained in the shoe. When walking or running, the foot heel is subject to a larger impact from ground. Therefore, the thickness of the rear heel section 123 is designed to be greater than that of the front metatarsal section 121. For example, the thickness of the front metatarsal section 121 is 2-3 mm, and the thickness of the rear heel section 123 is 7-8 mm, so the ground impact is able to be absorbed by the thicker part of the soft and elastic layer 1.

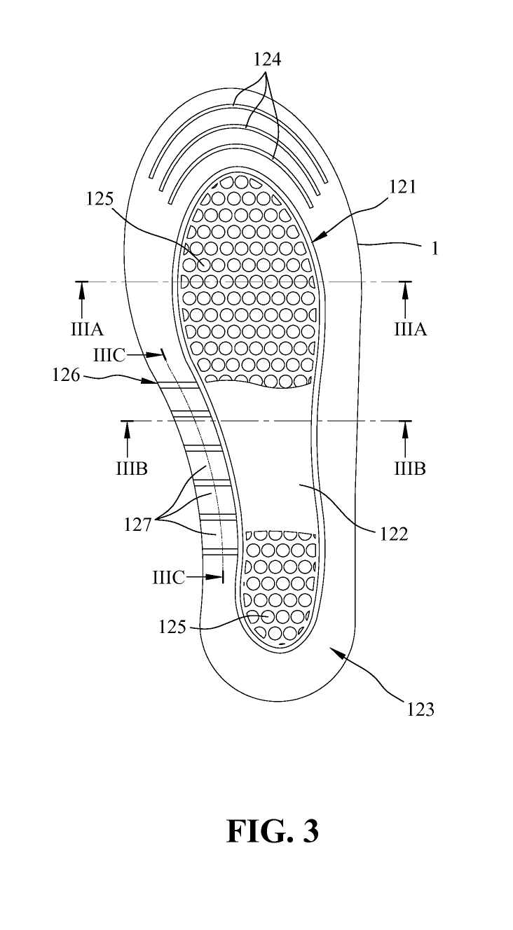

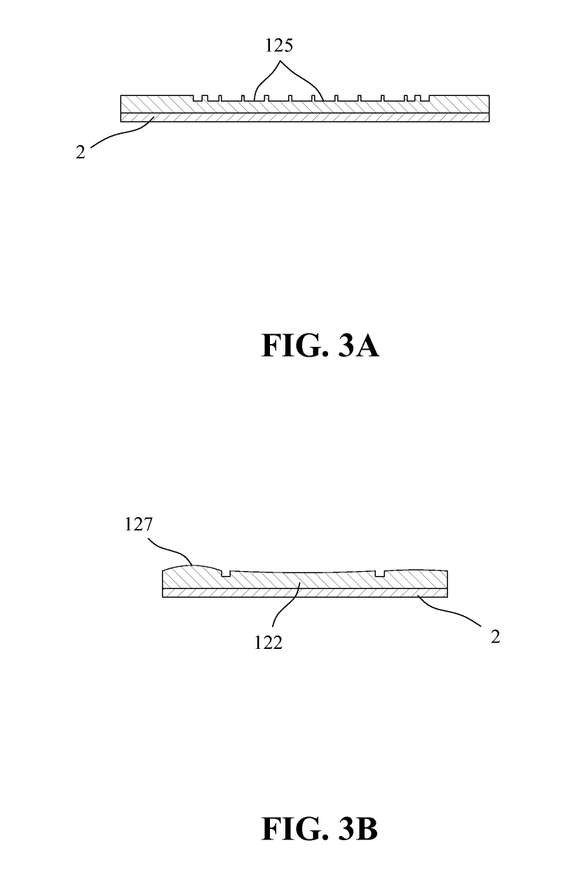

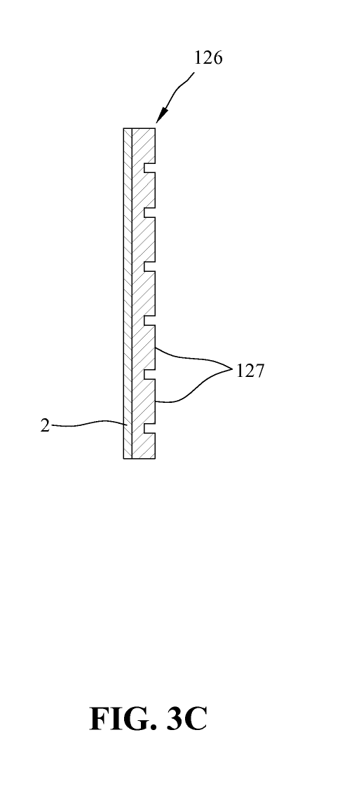

Referring to FIG. 3, the front end of the front metatarsal section 121 may be extended forward and the surface of the extended soft and elastic layer 1 has a plurality of arc-shaped marking lines 124 corresponding to different shoe sizes, so a user can tailor the shoe pad according to his foot size. FIGS. 3a, 3b and 3c are cross sectional views taken along lines 3A-3A, 3B-3B and 3C-3C of FIG. 3, respectively. Central portions of the front metatarsal section 121 and the rear heel section 123 include a plurality of cavities 125 distributed on the bottom surface 12 of the soft and elastic layer 1. The cavities 125 can enhance the flexibility of parts of the shoe pad that are often in a bended status so as to prolong the service life, and relieve the stress concentration generated when a user is standing still. The shapes and depths of the cavities 125 are not limited by the present invention. However, the depth is preferred to be less than 1 mm. Therefore the soft and elastic layer 1 where the cavities 125 is distributed is not too thick to ensure stable walking. The cavities 125 according to the embodiment of the present invention are circular cavities that are discrete from each other. The cavities 125 can also be shaped in other geometric shapes. The central portion of the rear heel section 123 having the cavities 125 is thinner than the periphery portion of the rear heel section 123. Therefore, when stepping on the central portion of the rear heel section 123, the foot heel feels descent.

A medial side of the mid foot section 122 includes an arch portion 126. The arch portion 126 is thicker than other areas of the shoe pad. The arch portion 126 has a plurality of reinforcement structures 127 for enhancing the arch supporting strength. The transversally arranged reinforcement structures 127 are protruded from a bottom surface of the arch portion 126 and have gaps therebetween. The arch portion 126 of the shoe pad according to the present invention is mainly served to support the longitudinal arch of foot so as to keep a certain radian of the foot arch. So when the foot steps on the ground, a proper support is provided, and an elastic forward moving assistance for walking is also provided, so the walking movement can be achieved without over using muscle strength and the muscle fatigue is greatly reduced.

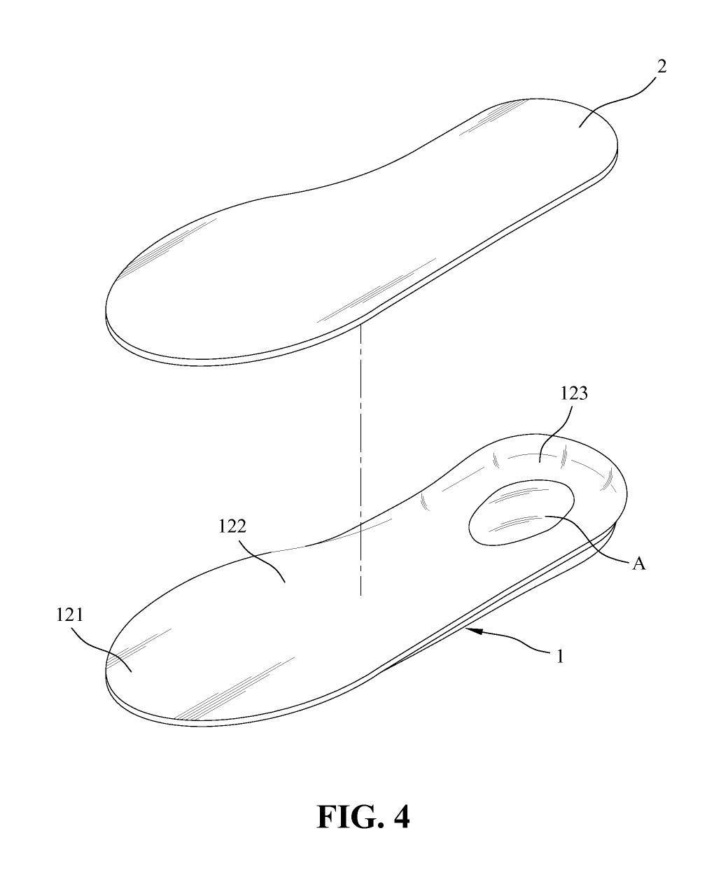

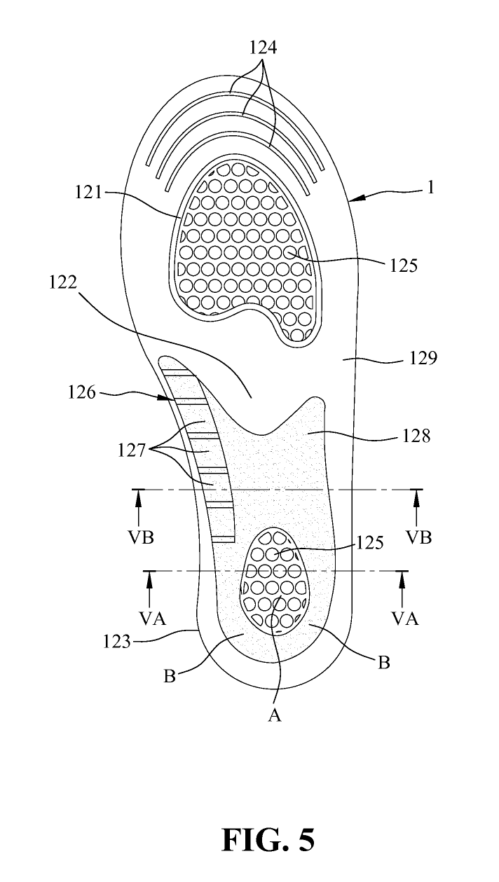

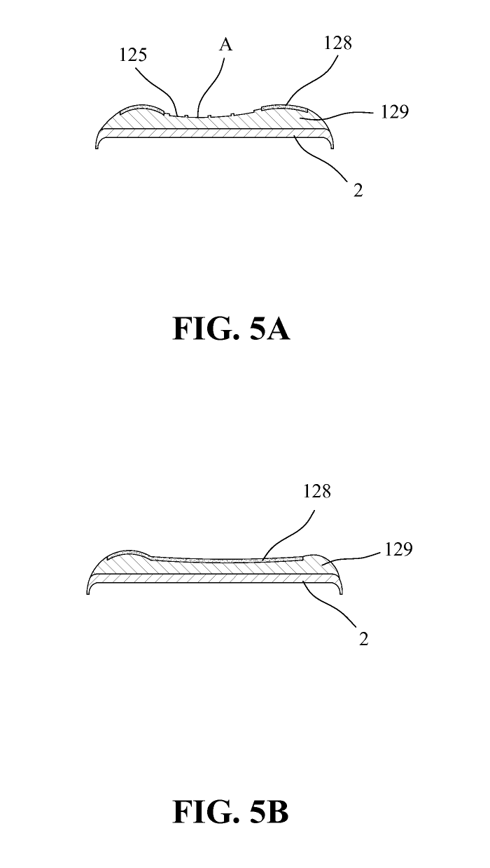

FIG. 4 is an exploded view of a soft and elastic shoe pad according to a second preferred embodiment of the present invention and FIG. 5 is a bottom view of the soft and elastic shoe pad of FIG. 4. FIGS. 5A and 5B are cross sectional views taken along lines 5A-5A and 5B-5B of FIG. 5, respectively. The central portion (area A) of the rear heel section 123 is curved upward, and the periphery portion (area B) of the rear heel section 123 is thicker than the central portion (area A) of the rear heel section 123. The rear heel section 123 is elastic and therefore the central portion (area A) of the rear heel section 123 is capable of descending when subject to pressure and resuming to the original state when the pressure is relieved. When the foot heel steps on the rear heel section 123, the central portion (area A) of the rear heel section 123 descends and the inner and outer rotation angles of the foot heel during walking can be effectively and stably controlled. Moreover, the soft and elastic layer 1 according to the first embodiment is integrally formed, and the soft and elastic layer 1 according to the second embodiment provides a manufacturing method different from that of the first embodiment. The method is to form an additional soft and elastic layer 128 corresponding to a region defined between the arch portion 126 and the periphery portion (area B) of the rear heel section 123, as shown in the shaded area of FIG. 5. The additional soft and elastic layer 128 is attached to a main layer 129 of the soft and elastic layer 1. Thus, according to actual needs, the additional soft and elastic layer 128 may be made of a material different from that of the main layer 129. The additional soft and elastic layer 128 and the main layer 129 may also be made of a same material but have different softness. Compared to the former integral forming method, the soft and elastic layer 1 made by the latter method is softer and has a better pressure relieving function.

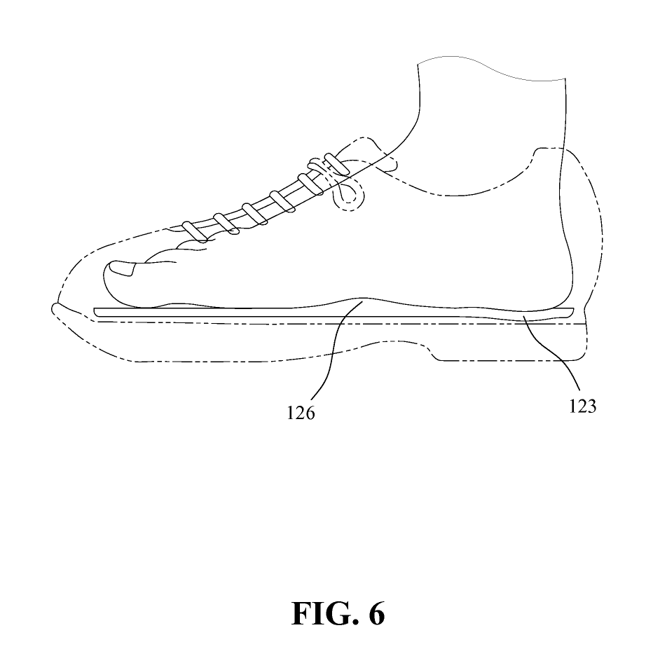

FIG. 6 is a side view illustrating that a foot steps on the shoe pad according to the second embodiment of the present invention. As mentioned above, one of characteristics of the shoe pad according to the present invention is the cavities 125 enhance the structural strength at the parts of shoe pad that are often in a bended status. Thus, fracture of the shoe pad is prevented and the stress concentration generated when a user is standing still is distributed over an area. The arch portion 126 of the shoe pad supports the medial longitudinal arch of foot, so when the foot steps on the ground, a proper support is provided, and an elastic forward moving assistance when walking is also provided, so the walking movement can be achieved without over using muscle strength, therefore the muscle fatigue is greatly reduced. When the foot heel is stepped on the rear heel section 123, the central portion (area A) of the rear heel section 123 descends; the inner and outer rotation angles of the foot heel during walking can be effectively and stably controlled.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A soft and elastic shoe pad, having a shape corresponding to an outline of a foot sole and disposed inside a shoe, comprising:
a soft and elastic layer including a front metatarsal section, a mid foot section and a rear heel section, central portions of the front metatarsal section and the rear heel section including a plurality of cavities distributed on a bottom surface of the soft and elastic layer, the cavities are discrete from each other, the mid foot section having a thickened arch portion, the arch portion including a plurality of reinforcement structures for enhancing the supporting strength; a periphery portion of the rear heel section is thicker than the central portion of the rear heel section, so when a foot heel steps on the rear heel section, a feeling of stable descending is formed; and
an air permeable layer connected onto a surface of the soft and elastic layer.

2. The shoe pad as claimed in claim 1, wherein a front end of the front metatarsal section is extended forward, and the extended front metatarsal section includes a plurality of marking lines corresponding to shoe sizes.

3. The shoe pad as claimed in claim 1, wherein the reinforcement structures are transversally arranged and protruded from a surface of the arch portion, and the reinforcement structures have gaps therebetween.

4. The shoe pad as claimed in claim 1, wherein the central portion of the rear heel section is curved upward.

5. The shoe pad as claimed in claim 1, wherein the soft and elastic layer includes a main layer and an additional soft and elastic layer attached onto the main layer, the additional soft and elastic layer is integrally formed and includes the arch portion and the periphery portion of the rear heel section.

6. The shoe pad as claimed in claim 5, wherein the main layer is made of silicone gel, latex, rubber, PU, TPE or EVA, and the additional soft and elastic layer is made of silicone gel, latex, rubber, PU, TPE or EVA.

7. The shoe pad as claimed in claim 1, wherein the air permeable layer is made of fabrics that are soft and capable of absorbing moisture.
